(19) United States
(12) Patent Application Publication
(10) Pub. No.: US 2020/0368149 A1
(43) Pub. Date: Nov. 26, 2020

(54) Topical Anesthetic Composition and Method of Synthesis

(71) Applicant: Wendy Johnson, La Vergne, TN (US)

(72) Inventor: Wendy Johnson, La Vergne, TN (US)

(21) Appl. No.: 16/856,236

(22) Filed: Apr. 23, 2020

Related U.S. Application Data
(60) Provisional application No. 62/851,984, filed on May 23, 2019.

(51) Int. Cl. A61K 9/00; A61K 31/445; A61K 9/06; A61K 47/18; A61K 47/14; A61K 47/10; A61K 47/22; A61K 47/02; A61K 47/44; A61K 47/34; A61K 36/738

(52) U.S. Cl. CPC A61K 9/0014; A61K 31/445; A61K 9/06; A61K 47/183; A61K 47/14; A61K 36/738; A61K 47/22; A61K 47/02; A61K 47/44; A61K 47/34; A61K 47/10

(57) ABSTRACT

A topical anesthetic composition for pain relief. The composition includes an anesthetic, such as bupivacaine, formulated for topical administration and transdermal delivery of the anesthetic. The composition can be applied to the skin of an individual to alleviate localized pain due to acute or chronic injuries or health conditions.

### CROSS REFERENCE TO RELATED APPLICATIONS

This application claims priority under 35 U.S.C. § 119(e) to U.S. Provisional Application No. 62/851,984 filed on May 23, 2019. The above identified patent application is incorporated by reference herein in its entirety to provide continuity of disclosure.

### BACKGROUND OF THE INVENTION

The present invention relates to a topical anesthetic composition for alleviating pain due to acute or chronic injuries or health conditions.

Many individuals experience painful procedures or events, whether surgical or non-surgical in nature, and suffer acute or chronic pain. Exemplary painful events include rotator cuff surgery, knee replacement, bullet wound repair, and the like. Oftentimes, the pain experienced is very severe, and may prevent the ability of the individual to rest or relax, which may prolong recovery. Some individuals may utilize systemic pain medications, but these substances may introduce adverse side effects or cause addiction. In addition, some individuals may have an aversion to needles used for injection of pain medications. A topical pain-relief composition may be advantageous for at least these individuals.

Therefore, there is a need in the art for a topical anesthetic composition with little or no adverse effects, such as addiction or needle fright, resulting from use. The present invention addresses this unmet need.

Substances have been disclosed in the art that relate to pain relief. These include compositions that have been patented and published in patent application publications. These compositions are often unsatisfactory and may lead to addiction. In view of the compositions disclosed in the art, it is submitted that there is a need in the art for an improvement to existing pain relief compositions. In view of the present disclosure, it is submitted that the present invention substantially diverges in structural and functional elements from compositions in the art, and substantially fulfills an unmet need in the art.

### SUMMARY OF THE INVENTION

In view of the disadvantages inherent in the known types of pain relief compositions in the art, the present invention provides a new and improved topical anesthetic composition, wherein the same can be utilized for relieving pain due to injury, illness, and the like.

It is therefore an object of the present invention to provide a topical anesthetic or pain-relief composition for relieving localized bodily pain.

In one aspect, the invention provides a topical anesthetic composition, comprising bupivacaine and a topical cream solution, wherein the topical cream solution configures the bupivacaine for topical application.

In another aspect, the invention provides a topical anesthetic composition, comprising bupivacaine, and a plurality of ingredients, wherein the plurality ingredients comprises a preservative, a humectant, an emulsifier, an emollient, a thickener, and a biocide. In such embodiments, the concentration of bupivacaine is about 122 nM, and one or more ingredients of the plurality of ingredients configures the bupivacaine for topical application.

In yet another aspect, the invention provides a method of synthesizing a topical anesthetic composition, comprising: combining an amount of bupivacaine with an amount of a topical cream solution; and stirring the amount of the bupivacaine and the amount of the topical cream solution until a homogenous cream is produced.

In various embodiments, bupivacaine concentration is about 122 nM. In some embodiments, the topical cream solution comprises a preservative, a humectant, an emulsifier, an emollient, a thickener, and a biocide.

In some embodiments, the preservative is selected from a group including, but not necessarily limited to: methylparaben, phenoxyethanol, disodium ethylenediaminetetraacetic acid (EDTA), methylisothiazolinone, iodopropynyl butylcarbamate (IPBC), and a combination thereof.

In some embodiments, the humectant is selected from a group including, but not necessarily limited to: glycerol, glyceryl stearate, and a combination thereof.

In some embodiments, the emulsifier is selected from a group including, but not necessarily limited to: cetyl alcohol, magnesium aluminum silicate, lecithin, polyacrylic acid (PAA), and a combination thereof.

In some embodiments, the emollient is selected from a group including, but not necessarily limited to: cetyl alcohol, cetyl acetate, sunflower seed oil, acetylated lanolin alcohol, and a combination thereof.

In some embodiments, the thickener is selected from a group including, but not necessarily limited to: a polyethyl ester, cetyl alcohol, PAA, magnesium aluminum silicate, and a combination thereof.

In some embodiments, the topical cream solution further comprises a fragrance additive selected from a group including, but not necessarily limited to: ethylene brassylate, sandalwood oil, rosa dascena extract, vanilla planifolia fruit extract, and a combination thereof.

Another object of the present invention is to provide a topical cream solution that may be readily manufactured from materials that permit relative economy and are commensurate with durability.

Other objects, features and advantages of the present invention will become apparent from the following detailed description taken in conjunction with the accompanying drawings.

### DETAILED DESCRIPTION OF THE INVENTION

The present invention provides a composition comprising an anesthetic, such as bupivacaine, that is formulated for topical delivery. Upon topical delivery, all or a portion of the anesthetic is absorbed through at least one layer of skin of an individual. In this manner, the anesthetic, which may ordinarily only be able to be administered through another route of delivery, such as by injection, is able to be administered to a specific site of the individual's skin for localized alleviation of pain.

Permeation of the anesthetic through the skin may be achieved by one or more compounds, such as carriers, that directly or indirectly interact with the anesthetic and facilitate absorption into or through the skin. These carriers may include, for example, hydrophilic and/or lipophilic compounds, and/or amphiphilic compounds, and may be formulated with other compounds to form a cream solution such as a topical cream solution, a gel such as a topical gel, a liquid such as a topical liquid, and the like. In this manner, a composition that comprises the anesthetic and one or more suitable carriers may effectively transform the anesthetic from being unable to be administered topically to being able to be administered topically.

In the exemplary embodiment of the present invention, the anesthetic comprises bupivacaine. Bupivacaine is an amide group anesthetic utilized for prevention of pain or discomfort, is ordinarily delivered to an individual by injection, and is unable to be administered topically. The present invention provides a composition comprising bupivacaine that is formulated for topical administration.

In various embodiments, the bupivacaine is combined with a topical cream solution to form a topical anesthetic composition that formulates the bupivacaine for topical administration. The bupivacaine may be present in the composition in an effective concentration.

In various embodiments, the effective concentration of bupivacaine is between 50-500 nM, between 75-250 nM, between 100-150 nM, about 122 nM, and/or 122 nM. In this manner, the anesthetic is present at a concentration that is suitable for alleviation of pain while also causing little or no side effects due to the medication.

The topical cream solution may comprise one or more of a preservative, a humectant, an emulsifier, an emollient, a thickener, and a biocide. The preservative prevents decay of the anesthetic of the composition. The humectant retains or preserves moisture within the composition so as to maintain its ability to be effectively applied to the skin for topical administration. The emulsifier stabilizes an emulsion of the composition, and may prevent separation of hydrophobic compounds from hydrophilic compounds in the composition. The emollient softens or smooths the skin when applied thereto. The thickener increases the viscosity of the composition such that the composition does not flow about or run off the skin before permeation therethrough. The biocide interferes with the adherence or growth of organisms, such as harmful organisms, within the composition, e.g., during preparation, storage, and use.

The preservative may include one or more of methylparaben, phenoxyethanol, disodium ethylenediaminetetraacetic acid (EDTA), methylisothiazolinone, and iodopropynyl butylcarbamate (IPBC). In some embodiments, the composition comprises methylparaben, phenoxyethanol, disodium ethylenediaminetetraacetic acid (EDTA), methylisothiazolinone, and iodopropynyl butylcarbamate (IPBC). In this manner, a plurality of preservatives may be utilized to maximize the time during which the anesthetic is active and present in sufficient concentrations to cause a medicinal effect during use.

The humectant may include one or more of glycerol, and glyceryl stearate. In some embodiments, the composition comprises glycerol and glyceryl stearate. In this manner, moisture may be maximally retained within the composition to configure the composition for topical application.

The emulsifier may include one or more of cetyl alcohol, magnesium aluminum silicate, lecithin, and polyacrylic acid (PAA). In some embodiments, the composition comprises cetyl alcohol, magnesium aluminum silicate, lecithin, and polyacrylic acid (PAA). In this manner, the emulsion of the composition may be maximally stabilized and separation of components of the composition is prevented from occurring.

The emollient may include one or more of cetyl alcohol, cetyl acetate, sunflower seed oil, and acetylated lanolin alcohol. In some embodiments, the composition comprises cetyl alcohol, cetyl acetate, sunflower seed oil, and acetylated lanolin alcohol. In this manner, the skin may be maximally softened or smoothed during use of the composition, and delivery of the active pharmaceutical ingredient, i.e., the anesthetic compound, may be effectively achieved along with treatment of the skin with the emollient.

The thickener or thickening agent may include one or more of a polyether ester, cetyl alcohol, PAA, and magnesium aluminum silicate. In some embodiments, the composition comprises a polyether ester, cetyl alcohol, PAA, and magnesium aluminum silicate. In this manner, a thickness of the composition may be suitable so as to ensure it is maintained in form and may easily be applied to the skin for topical administration of the anesthetic compound.

In some embodiments, the composition further comprises a fragrance additive to improve a fragrance or scent of the composition or a component thereof. Such fragrant additives may include, but may not necessarily be limited to, ethylene brassylate, sandalwood oil, rosa dascena extract, and vanilla planifolia fruit extract. In some embodiments, the composition comprises ethylene brassylate, sandalwood oil, rosa dascena extract, and vanilla planifolia fruit extract. In this manner, the scent of the composition may be aromatic or pleasant.

In various embodiments, the anesthetic is combined with the topical cream solution to form the composition of the present invention. In some embodiments, the topical cream solution comprises glycerol, petroleum jelly, mineral oil, stearic acid, glyceryl stearate, glycol stearate, polyethylene glycol (PEG-40) stearate, cetyl alcohol, cetyl acetate, sodium hydroxide, fragrance, dimethicone, phenoxyethanol, PAA, sunflower seed oil, disodium EDTA, acetylated lanolin alcohol, methylisothiazolinone, IPBC, aloe vera leaf juice, magnesium aluminum silicate, lecithin, borage seed oil, cholesterol, ascorbyl palmitate, sweet almond oil, ethylene brassylate, sandalwood oil, rosa damascene extract, and vanilla planifolia fruit extract. Accordingly, in such embodiments, a combination of ingredients including preservatives, humectants, emulsifiers, emollients, thickeners, and biocides are included in the topical cream solution to maximize an effectiveness and/or a utility thereof for topical administration or application.

In some embodiments, additional ingredients are included in the composition to improve solubility, stability, or effectiveness of one or more components or ingredients thereof. In such embodiments, sodium chloride (NaCl) may be present in a suitable amount so as to make the composition, or a precursor thereof, isotonic. In this manner, a concentration of ions in the composition is optimized.

To synthesize the composition of the present invention, a method of synthesizing the composition may be employed. In various embodiments, the method comprises combining an amount of bupivacaine with an amount of a topical cream solution, and stirring the amount of the bupivacaine and the amount of the topical cream solution until a homogenous cream is produced. In some embodiments, the concentration of bupivacaine in the topical anesthetic composition is about 122 nM. In some embodiments, the amount of bupivacaine is 2.5 mg of a 0.25% bupivacaine solution, and the amount of the topical cream solution is 6 fluid oz. In this manner, the composition may be synthesized from starting materials when needed, or alternately, may be synthesized when not needed and stored under refrigeration until needed.

The foregoing descriptions of specific embodiments of the present invention have been presented for purposes of illustration and description. They are not intended to be exhaustive or to limit the present invention to the precise forms disclosed, and modifications and variations are possible in view of the above teaching. The exemplary embodiment was chosen and described to best explain the principles of the present invention and its practical application, to thereby enable others skilled in the art to best utilize the present invention and its embodiments with modifications as suited to the use contemplated.

It is therefore submitted that the present invention has been shown and described in the most practical and exemplary embodiments. It should be recognized that departures may be made which fall within the scope of the invention. With respect to the description provided herein, it is submitted that the optimal features of the invention include variations in size, materials, shape, form, function and manner of operation, assembly, and use. All structures, functions, and relationships equivalent or essentially equivalent to those disclosed are intended to be encompassed by the present invention.

I claim:

1) A topical anesthetic composition, comprising:
bupivacaine, and a topical cream solution;
wherein the topical cream solution formulates the bupivacaine for topical application.

2) The composition of claim 1, wherein bupivacaine concentration is about 122 nM.

3) The composition of claim 1, wherein the topical cream solution comprises a preservative, a humectant, an emulsifier, an emollient, a thickener, and a biocide.

4) The composition of claim 3, wherein the preservative is selected from a group consisting of: methylparaben, phenoxyethanol, disodium ethylenediaminetetraacetic acid (EDTA), methylisothiazolinone, iodopropynyl butylcarbamate (IPBC), and a combination thereof.

5) The composition of claim 3, wherein the humectant is selected from a group consisting of: glycerol, glyceryl stearate, and a combination thereof.

6) The composition of claim 3, wherein the emulsifier is selected from a group consisting of: cetyl alcohol, magnesium aluminum silicate, lecithin, polyacrylic acid (PAA), and a combination thereof.

7) The composition of claim 3, wherein the emollient is selected from a group consisting of:
cetyl alcohol, cetyl acetate, sunflower seed oil, acetylated lanolin alcohol, and a combination thereof.

8) The composition of claim 3, wherein the thickener is selected from a group consisting of:
a polyether ester, cetyl alcohol, PAA, magnesium aluminum silicate, and a combination thereof.

9) The composition of claim 3, wherein the topical cream solution further comprises a fragrance additive selected from a group consisting of: ethylene brassylate, sandalwood oil, rosa dascena extract, vanilla planifolia fruit extract, and a combination thereof.

10) A topical anesthetic composition, comprising:
bupivacaine, and a plurality of ingredients;
wherein the plurality of ingredients comprises a preservative, a humectant, an emulsifier, an emollient, a thickener, and a biocide;
wherein bupivacaine concentration is about 122 nM;
wherein one or more ingredients of the plurality of ingredients formulates the bupivacaine for topical application.

11) The composition of claim 10, wherein the preservative is selected from a group consisting of: methylparaben, phenoxyethanol, disodium ethylenediaminetetraacetic acid (EDTA), methylisothiazolinone, iodopropynyl butylcarbamate (IPBC), and a combination thereof.

12) The composition of claim 10, wherein the humectant is selected from a group consisting of: glycerol, glyceryl stearate, and a combination thereof.

13) The composition of claim 10, wherein the emulsifier is selected from a group consisting of: cetyl alcohol, magnesium aluminum silicate, lecithin, polyacrylic acid (PAA), and a combination thereof.

14) The composition of claim 10, wherein the emollient is selected from a group consisting of: cetyl alcohol, cetyl acetate, sunflower seed oil, acetylated lanolin alcohol, and a combination thereof.

15) The composition of claim 10, wherein the thickener is selected from a group consisting of: a polyethyl ester, cetyl alcohol, PAA, magnesium aluminum silicate, and a combination thereof.

16) A method of synthesizing a topical anesthetic composition, comprising:
combining an amount of bupivacaine with an amount of a topical cream solution; and
stirring the amount of the bupivacaine and the amount of the topical cream solution until a homogenous cream is produced.

17) The method of claim 13, wherein the concentration of bupivacaine in the topical anesthetic composition is about 122 nM.

18) The method of claim 14, wherein the amount of bupivacaine is 2.5 mg of a 0.25% bupivacaine solution, and wherein the amount of the topical cream solution is 6 fluid oz.

19) The method of claim 15, wherein the topical cream solution comprises a preservative, a humectant, an emulsifier, an emollient, a thickener, and a biocide.

20) The method of claim 16, wherein the topical cream solution comprises glycerol, petroleum jelly, mineral oil, stearic acid, glyceryl stearate, glycol stearate, polyethylene glycol (PEG-40) stearate, cetyl alcohol, cetyl acetate, sodium hydroxide, fragrance, dimethicone, phenoxyethanol, PAA, sunflower seed oil, disodium EDTA, acetylated lanolin alcohol, methylisothiazolinone, IPBC, aloe vera leaf juice, magnesium aluminum silicate, lecithin, borage seed oil, cholesterol, ascorbyl palmitate, sweet almond oil, ethylene brassylate, sandalwood oil, rosa damascene extract, and vanilla planifolia fruit extract.
